(19) United States
(12) Patent Application Publication
(10) Pub. No.: US 2017/0035133 A1
(43) Pub. Date: Feb. 9, 2017

(54) ARTICLE AND PROCESS FOR COSMETICALLY ENHANCING EYEBROWS

(71) Applicant: Mark D. Ismach, Parkland, FL (US)

(72) Inventor: Mark D. Ismach, Parkland, FL (US)

(21) Appl. No.: 14/816,130

(22) Filed: Aug. 3, 2015

(51) Int. Cl. A41G 5/02; A45D 33/02; A61Q 19/00; A61K 8/34; A61K 8/92

(52) U.S. Cl. CPC A41G 5/02; A61K 8/342; A61K 8/92; A61K 8/345; A61Q 19/007; A45D 33/02; A61K 2800/20

(57) ABSTRACT

The invention is a process and a composition that provides an adhering composition for attaching colored fibrils to the eyebrows, the scalp and other skin covered regions. The composition is a rosewater glycerin mixture. The colored fibrils are short hair-like fibers of gossypium herbaceum, and they are applied after a sprayed mist of the composition is coated on the region that is to receive the fibrils. The fibrils color and increase the volume of the existing hair, and cover voids in the eyebrows, the scalp and other regions. When dry, the adhered colored fibrils are resistant to water. The fibrils are loose, and have a triboelectric negative charge and electrostatically attracted to hair and skin, which has a triboelectric positive charge. In the process, the fibrils are dabbed on the pre-wetted region, and any excess fibrils are removed. Eyebrows are sculpted to the desired color, thickness and shape.

### BACKGROUND OF THE INVENTION

1. Field of the Invention

The invention relates generally to an article of manufacture having a composition and a process for cosmetically enhancing thinning eyebrows and head hair.

2. Background

A number of techniques are used to shape eyebrows including plucking, waxing, and threading. With threading, a tensioned crossed-string is pulled over edges of the eyebrow at an angle of a desired line there plucking or trimming one or eyebrow hairs. Waxing can be a less precise technique as wax doesn't necessarily flow where it is anticipated, and plucking is subject to error as it is often not performed by a professional. In general, a problem with all the techniques is that eyebrow hairs do not necessary all grow with a predictable grain/angle, and instead of forming the desired line, the removal of eyebrow hairs can create voids. The voids can be cosmetically repaired with eyebrow pencils or brushed on mascara, but both techniques can smear or build-up or look slick, and in general do not produce a natural hair-like finish. Other solutions include dying and tattooing.

The prior art teaches that thinning or balding regions can be cosmetically enhanced by sprinkling on fibers to build hair volume and by coloring the scalp to match the hair. A problem with the prior art is that the user can accidentally remove the fibers by simply running their hands through their hair. Some permanence can be imparted with the application of a hair spray, but human skin can generate a high enough static electric charge to pull the fibers onto their hands, modifying the cosmetic appeal and putting detritus on their hands. The scalp is sometimes colored, but produces an unnatural look. In general building volume of existing hair, gives the most natural appearance.

The prior art does not teach utility of a process wherein eyebrows and thinning of balding regions are enhanced by simultaneously increasing the volume of existing hair, adding hair-like fibers to and coloring of eyebrows and other hair growth regions with a hair-like material is needed.

### SUMMARY OF THE INVENTION

The invention is a process wherein eyebrows having voids or thinning regions can be cosmetically enhanced by simultaneously adding colored fibers to an eyebrow and any void regions that require both color and hair-like fibers to create an overall desired shape, thickness and color. The invented process can be also said for other thinning or balding regions, for example a person's scalp.

The invention includes a composition that facilitates fastening a plurality of fibrils to skin and to hair, wherein the fibrils are short hair-like fibers derived from gossypium herbaceum, selected to have a desired color, for example dark brown. The fibrils are loose, they have a triboelectric negative charge, and they are intrinsically in electrostatic attraction to the hair and skin, which have a triboelectric positive charge.

As aspect of the invention is that the composition includes rose-water. Rose-water, a type of floral water, is a product of steeping rose petals in water or a hydrosol of essential oils and water soluble components extracted from rose sepals and petals. The rose-water nominally includes at least one moisturizer that increases the skin's hydration (water content). The moisturizer can include lightweight oils and other moisturizing constituents. The composition of the rose-water can vary as a consequence of the extraction process employed to generate the rose-water and the particular type of rose. Examples of roses that are strongly scented and historically used include Rosa damascene and Rosa centifolia.

Another aspect of the composition is that it includes glycerin. Glycerin is a lubricant and a humectant. It is frequently an ingredient in hair conditioners, and in high concentrations it can make hair slightly sticky for a period of time, and as will be discussed this characteristic can be advantageous.

Another aspect of the invention is that the composition enables a plurality of colored fibrils to be quickly applied to the eyebrows and other thinning or balding regions. Loose fibrils are distributed on and intermeshed with the existing hair when the fibrils are dabbed onto a region of hair and skin after being wetted with the composition. The fibrils are applied utilizing a dry brush, for example a foundation brush having a slanted flat tip, which is loaded with the loose fibrils by dipping the tip of the brush in a dispensed quantity of loose fibrils. Dabbing the brush causes the fibrils to transfer from the brush to the wetted regions, as the triboelectric negative charged fibrils are hydrophilically drawn to the composition and the triboelectric positively charged hair and skin. The transferred fibrils attach to and thicken existing eyebrow hair and form a hair-like texture on skin void of hair. Following drying, the attached fibrils are resistant to water, but can be removed using conventional shampoos, or with a heavy application of the invented composition.

The process for cosmetically enhancing eyebrows to create an overall desired shape, thickness and color; and to the scalp to augment the volume, coverage, and color includes the following steps: Applying, to clean skin, the composition as a sprayed mist, for example using a hand held nebulizer to generate the mist, and wiping down the eyebrows smoothing down any droplets. To a shallow container, shaking out a quantity of fibrils into a shallow container, therein refreshing the triboelectric negative charge on the quantity of fibrils. Loading a tip of a brush with the fibrils and applying the loose fibrils to a region of existing hair and skin that is to be thickened and colored. Removing unwanted excess applied fibrils. Sculpturing and the region to have the desired shape, thickness and uniformity. Grooming as required.

Optionally, the process can include drying the hair with a blow dryer. Also, the color can be augmented using for example to darken the color, applying mascara using a brush or a pencil for definition. The process can include applying a setting coating selected from clear mascara or a hair spray to augment the set.

Another aspect of the invention is that the composition can be applied utilizing a template of the region, such as a particular eyebrow template, which is positioned as a mask when spraying on the mist of the composition.

Another aspect of the invention is that it can be provided as an article of manufacture.

### BRIEF DESCRIPTION OF THE DRAWINGS

The foregoing invention will become readily apparent by referring to the following detailed description and the appended drawings in which:

FIG. 1 is illustrates the process steps for cosmetically enhancing thinning eyebrows.

### DETAILED DESCRIPTION OF THE INVENTION

The composition includes rose-water, glycerin and water. Some rose-water already has glycerin, so the relative amounts would need to be adjusted to compensate for this adulterant. A working formulation has by volume about 1 part rose-water, about ½ part glycerin, and about ½ part water. For an about 100 milliliter bottle with a pump nebulizer, a workable volume is about 40 milliliters rose-water, about 20 milliliters glycerin, and about 20 milliliters water. The composition should be mixed on addition of the components, and shaken just prior to spraying.

The composition has a high volume of glycerin for the volume of rose-water, and produces unanticipated results. Glycerin is miscible with water, and the Applicant asserts that the addition of a high volume of glycerin causes the hydrosol to become less stable, which ultimately results in a faster stronger bond between the fibrils and the eyebrow hair and skin. When the glycerin is admixed with the rose-water, the glycerin is taken-up by the suspended rose-water phase as well as the water phase, causing the suspended rose oil in the rose-water phase to swell with glycerin to a point where some of the rose oil separates, and the composition has to be shaken to disperse the phases when the composition sprayed. The glycerin is distributed between the rose-water phase and the water phase. The shaking prior to use maintains a uniform dispersion of the phases. The admixture of rose-water oil and glycerin is very similar to the oils found naturally on hair and skin, and the glycerin functions to quickly wet the hair and skin with the rose oils. The fibrils too have naturally have oils (cotton seed oil) and the glycerin functions to quickly wet the fibrils, when they are applied. The result is the fibrils are quickly adhered to the already wetted eyebrow hair and skin.

The composition facilitates the initial application of the fibrils and as the water and light alcohols evaporate the attachment is strengthened. As previously noted the fibrils are short hair-like fibers derived from gossypium herbaceum, wherein short is defined as having a length of about 0.1 millimeters to about 1.5 millimeters. The fibrils that are selected to be used have a desired color, for example dark brown, black, or another color that cosmetically enhances an application region. The fibrils are loose, having a triboelectric negative charge, and they are intrinsically in electrostatic attraction to the hair and skin, which have a triboelectric positive charge. The fibrils can be shaken out of a dispenser, which nominally is a bottle with a perforated lid. Dispensing tends to increase the voltage of the triboelectric negative charge.

In one embodiment the rose-water has at least one moisturizer, wherein the moisturizer increases the skin's hydration. An effective moisturizer includes lightweight oils, such as cetyl alcohol, and other moisturizing constituents. Rose-water is predominately water, and in addition to water, it typically has citronellol, nonadecane, geraniol and phenyl ethyl alcohol, and also henicosane, 9-nonadecen, eicosane, linalool, citronellyl acetate, methyleugenol, heptadecane, pentadecane, docosane, nerol, disiloxane, octadecane, and pentacosane. The phenyl ethyl alcohol imparts much of the fragrance. The exact content will vary as a consequence of the process of how the rose petals and sepals are extracted and the particular species of rose. Previously Rosa damascene and Rosa centifolia were cited as being exemplary.

Glycerin, also called glycerol, is odorless and it is a lubricant and a humectant. It has a boiling point of about 550 F, and so it will not evaporate, but it can be absorbed by hair and skin, and it is miscible with water and many floral oils. It is frequently found in hair conditioners, and in high concentrations it will make hair and cotton derived fibers like gossypium herbaceum slightly sticky for a period of time. The applicant has observed that as the water and lighter components of the composition evaporate the bond of the fibrils to the hair and the skin gets stronger. Evidence of this is that when the composition has been freshly applied, the fibrils can be easily shaped and wiped away, but on becoming dry, the bonds of the fibrils to skin, and particularly the hair, are resistant to dissolution by water.

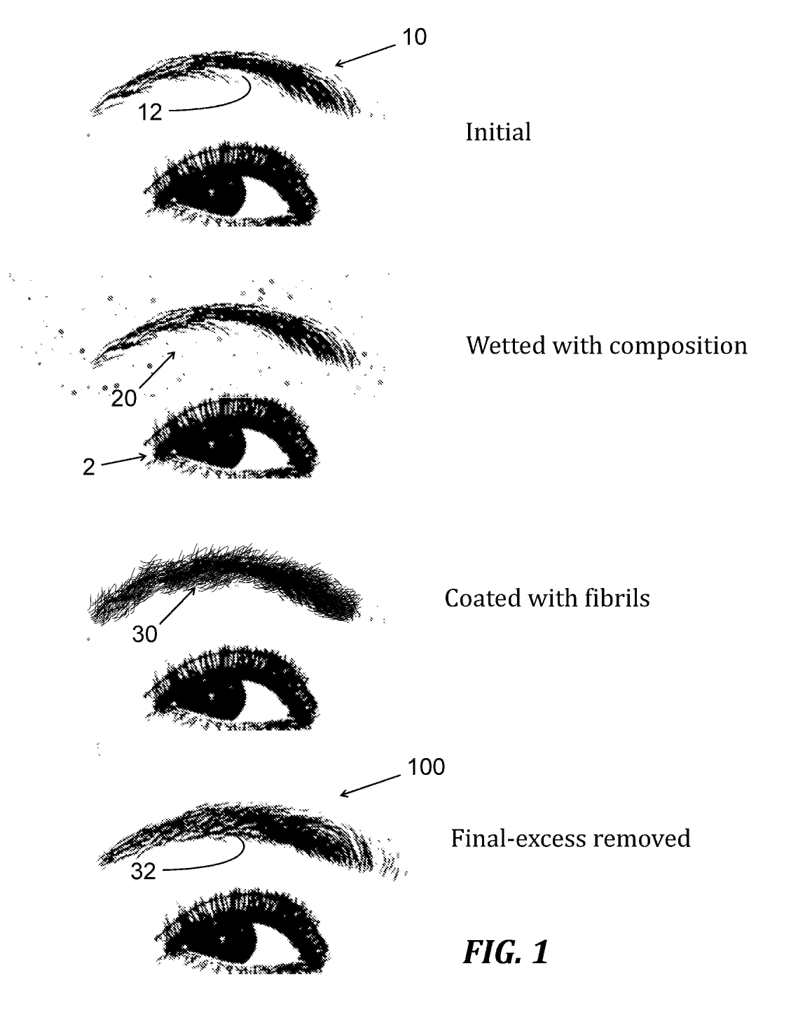

FIG. 1 illustrates the invented process of wherein the fibrils are applied to hair and skin, and more specifically to cosmetically enhance eyebrows. In the initial step, the user's eyebrow is clean and dry, having no makeup or colorant. In the figure, the eye and eyebrow hair are indicated by black lines and the skin is indicated by an absence of lines. Various skin and hair colors can be inferred by the reader.

As shown in a first step the eyebrow 10 has been cleaned, exposing a void region 12, where there is very little eyebrow hair.

In a second step a mist of the shaken composition 20 has been spayed onto the eyebrow and a proximate region. The mist has formed droplets, indicated by a plurality of dots above the eye 2. Following application of the composition, the eyebrow is smoothed down to ensure uniform coverage over the entire eyebrow and to wipe off any excess composition.

In next step, the colored fibrils 30 are dabbed onto the wet eyebrow. Colored fibrils are applied utilizing a dry brush, for example a foundation brush having a slanted flat tip, which is loaded with a plurality of loose fibrils by dipping the tip in a dispensed quantity of loose fibrils. Dabbing the brush causes the fibrils to transfer to the eyebrow and the wet proximate region, as the triboelectric negative charged fibrils are drawn to the composition and the triboelectric positively charged hair and skin. The applied fibrils thicken the existing hair and adhere to void areas forming a hair-like texture on the skin. After dabbing, excess loose fibrils are wiped off, therein sculpturing the fibril filled eyebrow to have the desired shape, thickness, color and uniformity.

As shown in a last step the attached fibrils are filling in the finished eyebrow 100, which is now fibril filled making it thicker. The void area is now eliminated, covered with fibrils 32.

The finished eyebrow has the desired shape, thickness, color and uniformity; but needs a period of time to set. Normally only a couple of minutes are required for an eyebrow. More time for heavier applications over larger regions. Grooming and tapering are possible as required. Drying can be accelerated by applying low velocity blow drying air. Additionally, a setting coat of clear mascara or a hair spray can optionally be applied. The finished eyebrow can be further colored with a mascara brush or a colored pencil.

The invention can be used with eyebrow templates, wherein the template is positioned as a mask when the composition is spayed on.

The invention is provided as an article of manufacture that includes a plurality of fibrils of short hair-like fibers derived from gossypium herbaceum, selected to have a desired color. Finally Hair™ offers at least sixteen colors. The colors include black, soft black, dark chocolate brown, dark brown, medium brown, light brown, light medium brown, red, auburn, blonde, blonde, medium blonde, golden blonde, dark blonde, grey, and white. The article further includes a brush having bristles which have an affinity for the fibrils; a composition that comprises a floral-water and glycerin; at least one dispenser for the colored fibrils; and a nebulizer for generating a sprayed mist of the composition; wherein the article enables a cosmetic modification of a region having hair and a region devoid of hair, as the article enables an increase in volume, uniformity of color, and coverage of the region having some hair, and a simulation of hair in the region devoid of hair. Examples of regions having thinning hair and regions devoid of hair include receding hair lines, bald patterns, and eyebrows that are discontinuous or overly thinned.

Finally, any numerical parameters set forth in the specification and attached claims are approximations (for example, by using the term “about”) that may vary depending upon the desired properties sought to be obtained by the present invention. At the very least, and not as an attempt to limit the application of the doctrine of equivalents to the scope of the claims, each numerical parameter should at least be construed in light of the number of significant digits and by applying ordinary rounding.

What is claimed is:

1. A composition that facilitates fastening a plurality of loose electrostatically charged colored fibrils to hair and skin, wherein the composition is an admixture comprised of a volume of about 1 part rose-water, about one half part glycerin, and about one half part water; where the glycerin is distributed between a rose-water phase and a water phase, and the admixture requires shaking prior to use to maintain a uniform dispersion of the phases throughout the composition.

2. The composition according to claim 1, wherein said rose-water is comprised of at least one moisturizer, wherein the moisturizer increases the skin's hydration.

3. The composition according to claim 1, wherein said rose-water is comprised of cetyl alcohol and lightweight oils.

4. An article of manufacture, said article comprises: a plurality of fibrils of short hair-like fibers derived from gossypium herbaceum, selected to have a desired color; a brush having bristles which have an affinity for the fibrils; a composition that comprises a floral-water and glycerin; at least one dispenser for the colored fibrils; and a nebulizer for generating a sprayed mist of the composition; wherein the article enables a cosmetic modification of a region having hair and a region devoid of hair, as the article enables an increase in volume, uniformity of color, and coverage of the region having some hair, and a simulation of hair in the region devoid of hair.

5. The article of manufacture according to claim 4, wherein the at least one dispenser contains fibrils of at least one color.

6. The article of manufacture according to claim 4, wherein the brush is a makeup foundation brush.

7. The article of manufacture according to claim 6, wherein the makeup foundation brush has a slanted flat tip.

8. The article of manufacture according to claim 4, wherein the floral-water is rose-water.

9. The article of manufacture according to claim 4, wherein the colored fibrils are selected from the consisting of black, soft black, dark chocolate brown, dark brown, medium brown, light brown, light medium brown, red, auburn, blonde, blonde, medium blonde, golden blonde, dark blonde, grey, white, and combinations thereof.

10. A process of simultaneously adding color and volume to an eyebrow, said process comprising the steps of:
cleaning and drying the eyebrow;
spraying a mist of a shaken composition comprised of rose-water and glycerin on the clean eyebrow;
wiping the clean eyebrow to distribute the composition throughout the eyebrow and to remove excess composition;
dabbing a plurality of loose fibrils of a selected color onto the eyebrow therein filling in the eyebrow and any void regions with the loose fibrils;
removing excess loose fibrils, therein sculpturing the fibril filled eyebrow to have the desired shape, thickness, color and uniformity; and
allowing a sufficient time for the fibril filled eyebrow to dry, wherein the fibrils are set, and the fibril filled eyebrow is a finished eyebrow.

11. The process according to claim 10, wherein the process can furthermore include:
positioning an eyebrow template as a mask when spraying the mist of the shaken composition.

12. The process according to claim 10, wherein the process can furthermore include:
applying a setting coat selected from a clear mascara, a hair spray, and a colored mascara.
